(19) United States
(12) Patent Application Publication
(10) Pub. No.: US 2002/0082306 A1
(43) Pub. Date: Jun. 27, 2002

(54) Aqueous formulation of beta carotene

(75) Inventor: Werner Josef Frantsits, Wien (AT)

Correspondence Address: YOUNG & THOMPSON, 745 SOUTH 23RD STREET 2ND FLOOR, ARLINGTON, VA, 22202

(73) Assignee: Sanochemia Pharmazeutika Aktiengesellschaft

(21) Appl. No.: 10/085,000

(22) Filed: Mar. 1, 2002

Related U.S. Application Data
(63) Continuation of application No. 09/460,769, filed on Dec. 14, 1999, now abandoned.

(30) Foreign Application Priority Data
Dec. 15, 1998 (AT) A 2092/98

(51) Int. Cl. A61K 31/015

(52) U.S. Cl. 514/763

(57) ABSTRACT

An aqueous formulation of beta-carotene that is suitable for use in veterinary medicine contains polyoxyethylene-660-hydroxystearate and/or isopropyl myristate as a mediator of solubility. The formulation, which for example contains 0.1-10% (w/v) beta-carotene, at least one antioxidant and at least one preservative can be additionally present. Further, a method for manufacturing the aqueous formulation of beta-carotene is described.

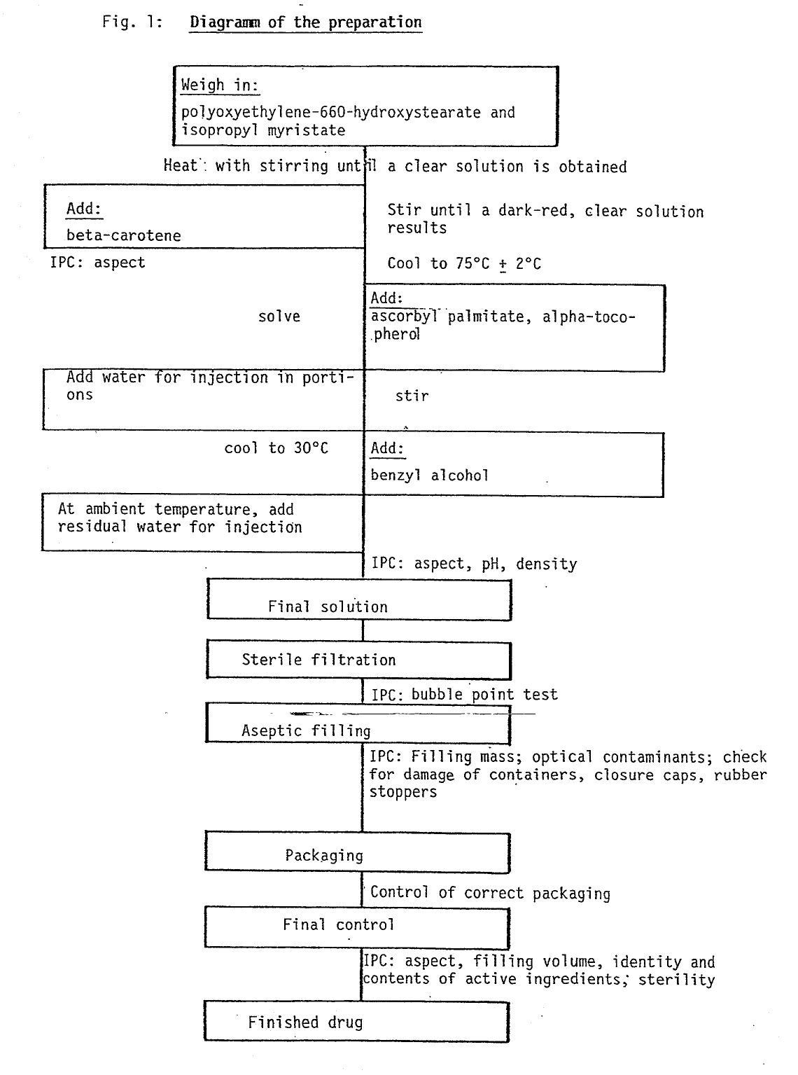

### FIELD OF THE INVENTION

The invention concerns an aqueous formulation of beta-carotene which is particularly suited for parenteral administration, and also a method for preparing said formulation.

### BACKGROUND OF THE INVENTION

Since the conditions of intensive utilization of animals for human consumption is frequently associated with a deficiency in beta-carotene that impairs reproduction, beta-carotene has been used in veterinary medicine for a long time, both as a feed supplement and particularly as injectable formulations for the rapid alleviation of acute deficiency syndromes.

The pharmacological effect of beta-carotene with respect to its fertility-enhancing properties, such as stabilization of the corpus luteum, the increase of plasma progesterone levels, and generally the maintenance of existing gravidity is known. It is also known that administration of beta-carotene improves the immune status of the offspring.

Injectable carotene is also used in the therapy of veterinary endometriosis, for instance in cattle, hors and dog.

Beta-carotene is practically insoluble in water as well as in the usually employed aqueous formulations. Beta-carotene is also hardly soluble in ethanol, cyclohexane and ether. An additional problem is that, in the presence of light and heat, dissolved beta-carotene is easily decomposed by oxygen from ambient air.

As a consequence, the above-mentioned properties of beta-carotene constitute a challenge on the galenic level because the bioavailability and stability that is demanded from a drug could not, or only insufficiently, be guaranteed.

During attempts at developing oral formulations, lipoid solutions (such as olive oil) have occasionally been employed either as such, or as aqueous emulsions.

Oily solutions of beta-carotene are however unsuitable for parenteral administration. Pure oil formulations penetrate into the blood stream only slowly, and in the meantime the unavoidable deposition of oil and beta-carotene at the injection site cause considerable pain and histopathological alterations for the animal.

Emulsions are problematic because their stability towards spontaneous phase separation is low. In addition, resistance of beta-carotene against oxydation by oxygen from the ambient air is further decreased in emulsions.

### OBJECT OF THE INVENTION

It is therefor the objective for the invention to provide an aqueous formulation of betacarotene that is in particular suitable for parenteral administration, as well as a method for the preparation of said formulation.

### SUMMARY OF THE INVENTION

This objective is met by a formulation according to claim 1 and, as far as the method is concerned, with a method of manufacture according to claim 10.

It is an essential characteristic of the invention that the formulation according to the invention contains beta-carotene as a micellar solution (micro-emulsion), in such a way that it retains unlimited utility (with respect to both the beta-carotene content of the formulation and its resistance against phase separation) for a period of at least two years if stored at normal ambient temperature and protected from light.

A formulation that is suitable for parenteral (in particular, intramuscular) administration that for example can be administered to hoof and claw animals as well as dogs can have a beta carotene content between 0.1% and 10% (w/v), in particular between 1 and 5% (w/v). Particularly, the micellar solution of beta-carotene according to the invention can contain a mixture of isopropyl myristate and polyoxyethylene-660-hydroxystearate in the aqueous medium, with a concentration of isopropyl myristate between 5 and 20% (w/v) and the concentration of polyoxyethylene-660-hydroxystearate between 10 and 40% (w/v). Concentrations of 5 to 10% (w/v) for isopropyl myristate and 15-20% (w/v) for polyoxyethylene-660-hydroxystearate are preferred.

The formulation according to the invention can contain at least one antioxidant, such as ascorbyl palmitate and/or DL-alpha-tocopherol. The antioxidant content can be 0.01 to 1.0% (w/v), 0.02-0.03% (w/v) being preferred.

### DETAILED DESCRIPTION OF THE INVENTION

A preferred method to manufacture a micellar solution of beta-carotene in an aqueous medium that is suitable for parenteral administration is explained in the enclosed block diagram which details an example of a preferred working protocol for manufacture. The acronym “IPC” that is used in the block diagram stands for “In-Process Control.”

In the first step of the method, polyoxyethylene-660-hydroxystearate and isopropyl myristate are weighed into water for injection in such an amount that the concentration of isopropyl myristate in the final preparation is 5-20% (w/v), and the final concentration of polyoxyethylene-660-hydroxystearate in the final preparation is 10-40% (w/v). This mixture is heated stirred until a clear solution results.

To the clear solution that has been obtained above an amount of beta-carotene is added that corresponds to a beta-carotene content of 0.1-10% (w/v) in the final solution is added with stirring. During the dissolution of beta-carotene the mixture is kept within a temperature range of 100-140° C. (118-128° C. being preferred). Stirring is continued until a dark-red, clear solution is obtained.

The solution that is obtained is cooled to 75° C. +/−2° C. Then, ascorbyl palmitate and DL-alpha-tocopherol are added as antioxidants, the amounts added being such that each antioxidant is present in an amount of 0.005-0.05% (w/v).

To the resultant mixture water for injection is added in portions under stirring, and after cooling to 30° C. benzyl alcohol is added as a preservative. After the addition of benzyl alcohol (10 mg/ml) the residual amount of water for injection is added at ambient temperature and the aspect, the pH and the density of the resultant micellar solution (micro-emulsion) is checked.

The resultant final solution is sterilized by filtration.

Sterile filtration is followed by aseptic filling, labeling and packaging followed by a final control with respect to aspect, filling volume, identity, content of the active ingredients and sterility.

The resulting product is a formulation of beta-carotene in an aqueous medium that is suitable for parenteral administration, and remains usable for a period of two years when stored at room temperature and protected from light.

The acronym “% (w/v)” stands for the mass of the respective substance, expressed as a percentage of the volume of the final aqueous preparation of beta-carotene.

An aqueous preparation of beta-carotene that can be used in veterinary medicine contains polyoxyethylene-660-hydroxystearate and/or isopropyl myristate as a mediator of solubility. In a preparation that contains, for example, 0.1-10% (w/v) beta-carotene, at least one antioxidant and at least one preservative can be additionally present.

What is claimed is:

1. A method for preparing a formulation of betacarotene in an aqueous medium, wherein the formulation contains at least polyoxyethylene-660-hydroxystearate and isopropyl myristate as a mediator of solubility and at least one of ascorbyl palmitate and alpha-tocopherol as an antioxidant, comprising heating an aqueous solution of polyoxyethylene-660-hydroxystearate to a temperature between 70° C. and 140° C., adding beta-carotene to the heated aqueous solution of polyoxyethylene-660-hydroxystearate with stirring, adding at least one of ascorbyl palmitate and alpha-tocopherol as antioxidant to the solution of polyoxyethylene-660-hydroxy-stearate and beta-carotene heated to a temperature of 75°C. +/−2° C., and diluting the solution thus obtained by adding water to make an injectable formulation containing 0.1-10% (w/v) beta-carotene, 10-40% (w/v) polyoxydrhylene-660-hydroxy-stearate and 5-20% (w/v) isopropyl myristate.

2. A method as claimed in claim 1, wherein the concentration of polyoxyethylene-660-hydroxystearate is 10-40% (w/v).

3. A method as claimed in claim 2, wherein said concentration is 15-20% (w/v).

4. A method as claimed in claim 1, wherein the beta-carotene content is 0.1-10% (w/v).

5. A method as claimed in claim 4, wherein said beta-carotene content is 1-5% (w/v).

6. A method as claimed in claim 1, wherein the solution contains isopropyl myristate as an additional mediator of solubility in a concentration of 5-20% (w/v).

7. A method as claimed in claim 6, wherein said concentration of isopropyl myristate is 5-10% (w/v).

8. A method as claimed in claim 1, wherein the concentration of the antioxidant is 0.01-1.0% (w/v).

9. A method as claimed in claim 8, wherein the concentration of the antioxidant is 0.02-0.3% (w/v).

10. A method as claimed in claim 1, wherein the concentration of ascorbyl palmitate and alpha-tocopherol each is 0.005-0.05% (w/v).

11. A method as claimed in claim 10, wherein said concentration of ascorbyl palmitate and alpha-tocopherol each is 0.01-0.15% (w/v).

12. A method as claimed in claim l, wherein following cooling to 30° C. +/−5° C. the solution containing polyoxyethylene-660-hydroxystearate, beta-carotene and at least one antioxidant is mixed with a preservative.

13. A method as claimed in claim 12, wherein said preservative is benzyl alcohol in an amount of 5 mg/ml.
